(19) United States
(12) Patent Application Publication
(10) Pub. No.: US 2026/0294880 A1
(43) Pub. Date: Oct. 1, 2026

(54) COMPOSITION FOR TREATING OTOTOXIC HEARING LOSS CAUSED BY AMINOGLYCOSIDE ANTIBIOTICS

(71) Applicants: KYUNGPOOK NATIONAL UNIVERSITY INDUSTRY-ACADEMIC COOPERATION FOUNDATION, Daegu (KR); KYUNGPOOK NATIONAL UNIVERSITY HOSPITAL, Daegu (KR); INDUSTRY-ACADEMIC COORPERATION FOUNDATION DAEGU HAANY UNIVERSITY, Gyeongsan-si, Gyeongsangbuk-do (KR)

(72) Inventors: Un-Kyung KIM, Seoul (KR); Ye-Ri KIM, Daegu (KR); Kyu-Yup LEE, Daegu (KR); Jeong-In BAEK, Daegu (KR)

(73) Assignees: KYUNGPOOK NATIONAL UNIVERSITY INDUSTRY-ACADEMIC COOPERATION FOUNDATION, Daegu (KR); KYUNGPOOK NATIONAL UNIVERSITY HOSPITAL, Daegu (KR); INDUSTRY-ACADEMIC COORPERATION FOUNDATION DAEGU HAANY UNIVERSITY, Gyeongsan-si, Gyeongsangbuk-do (KR)

(21) Appl. No.: 19/479,331

(22) PCT Filed: Apr. 22, 2024
(86) PCT No.: PCT/KR2024/005421

§ 371 Date: Oct. 27, 2025

(30) Foreign Application Priority Data
Apr. 28, 2023 (KR) 10-2023-0056472
Aug. 21, 2023 (KR) 10-2023-0108922

(51) Int. Cl. A61K 31/4375; A61K 31/7036; A61P 27/16

(52) U.S. Cl. CPC A61K 31/4375; A61K 31/7036; A61P 27/16

(57) ABSTRACT

The present invention relates to a pharmaceutical composition for preventing or treating hearing loss, the composition containing berberine chloride as an active ingredient, wherein the pharmaceutical composition was found to effectively inhibit hair cell apoptosis caused by reactive oxygen species and exhibit an excellent protective effect on hair cells, and thus can be effectively used as a drug or health supplement for preventing or treating hearing loss.

### TECHNICAL FIELD

The present disclosure provides a composition for treating an ototoxic hearing loss caused by an aminoglycoside antibiotic.

### BACKGROUND ART

Hearing loss (hearing impairment) is a disease that affects 15 to 20% of the entire population, with the number of people with hearing loss increasing due to environmental pollution and aging in modern society, and since hearing impairment once developed is permanent, it is very important to prevent it before development. Hearing loss is mostly caused by environmental factors and genetic factors such as sudden, drug-induced (anticancer drugs, antibiotics, etc.), noise-induced, traumatic, age-related, and congenital causes and is mainly due to damage and death of auditory cells. While most research on auditory cell damage has focused on noise-induced hearing loss, there is a scarcity of research on hearing loss caused by mechanisms of ototoxic drug-induced damage.

Aminoglycosides, a class of antibiotics, are known to be used in the treatment of infections by Gram-negative bacteria. Although it is the most widely used antibiotic due to its high efficacy and low cost, major side effects such as nephrotoxicity and ototoxicity have been reported. Since drug-induced ototoxicity is one of the major causes of acquired hearing loss, three types of aminoglycosides (amikacin, kanamycin, and gentamicin) can cause damage to cochlear hair cells, but, due to the lack of understanding in the ototoxic hearing loss caused by aminoglycoside drugs, the development of treatments therefor has been lagging, with no practical preventive drug at present.

### PRIOR-ART DOCUMENT

### Patent Document

(Patent Document 1) 1. Korean Patent Application Publication No. 10-2018-0020229

### DISCLOSURE OF THE INVENTION

### Technical Goals

An object of the present disclosure is to provide a pharmaceutical composition for preventing or treating an ototoxic hearing loss caused by an aminoglycoside drug, including berberine chloride as an active ingredient.

Another object of the present disclosure is to provide a combination of antimicrobial agents, including berberine chloride and an aminoglycoside drug.

Another object of the present disclosure is to provide a health functional food composition for preventing or ameliorating an ototoxic hearing loss caused by an aminoglycoside drug, including berberine chloride as an active ingredient.

### Technical Solutions

To achieve the above objects, the present disclosure provides a pharmaceutical composition for preventing or treating an ototoxic hearing loss caused by an aminoglycoside drug, including berberine chloride as an active ingredient.

In addition, the present disclosure provides a combination of antimicrobial agents, including berberine chloride and an aminoglycoside drug.

In addition, the present disclosure provides a health functional food composition for preventing or ameliorating an ototoxic hearing loss caused by an aminoglycoside drug, including berberine chloride as an active ingredient.

### Advantageous Effects

According to the present disclosure, since it has been determined that berberine chloride exhibits an effect of preventing and treating damage to auditory cells from ototoxic drugs such as aminoglycoside drugs, a composition including berberine chloride as an active ingredient may be provided as a pharmaceutical composition and health food for preventing or treating an ototoxic hearing loss.

### BRIEF DESCRIPTION OF DRAWINGS

FIG. 1 shows a protective effect of berberine chloride (hereinafter referred to as BC) on mouse hair cells from ototoxicity.

FIG. 2 shows an inhibitory effect on death of hair cells following BC treatment, induced by treatment of three aminoglycoside drugs [amikacin (hereinafter referred to as AK), kanamycin (hereinafter referred to as KM), and gentamicin (hereinafter referred to as GM)].

FIG. 3 shows an inhibitory effect of BC on accumulation of mitochondrial reactive oxygen species (ROS) in cochlear hair cells.

FIG. 4 shows a protective effect of BC on cochlear hair cells from treatment of aminoglycoside drug for the mitochondrial membrane potential.

### BEST MODE FOR CARRYING OUT THE INVENTION

Hereinafter, the present disclosure will be described in more detail.

The present disclosure provides a pharmaceutical composition for preventing or treating an ototoxic hearing loss caused by an aminoglycoside drug, including berberine chloride as an active ingredient.

The aminoglycoside drug may be selected from the group consisting of amikacin, kanamycin, and gentamicin, but is not limited thereto.

The pharmaceutical composition may inhibit apoptosis of auditory cells and increase the number of hair cells.

The pharmaceutical composition may inhibit accumulation of mitochondrial reactive oxygen species (ROS).

In other embodiments of the present disclosure, the pharmaceutical composition may further include one or more additives selected from the group consisting of appropriate carriers, excipients, disintegrators, sweeteners, coating agents, leavening agents, lubricants, glydents, flavoring agents, antioxidants, buffers, bacteriostatic agents, diluents, dispersants, surfactants, binders, and lubricants that are commonly used in the manufacture of pharmaceutical compositions.

Specifically, lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate, and mineral oil may be used as the carriers, excipients, and diluents, solid preparations for oral administration may include tablets, pills, acids, granules, and capsules, and these solid preparations may be prepared by mixing, in the composition, at least one or more excipients, for example, starch, calcium carbonate, sucrose or lactose, and gelatin. In addition to simple excipients, lubricants such as magnesium stearate and talc may also be used. Oral liquid preparations may include suspensions, liquids, emulsions, and syrups, and various excipients, such as humectants, sweeteners, fragrances, and preservatives may be included in addition to the simple diluents that are commonly used, such as water and liquid paraffin. Preparations for parenteral administration include sterile aqueous solutions, non-aqueous solvents, suspensions, emulsions, lyophilized agents, and suppositories. For non-aqueous solvents and suspensions, propylene glycol, polyethylene glycol, vegetable oils such as olive oil, and injectable esters such as ethyl oleate may be used. As for base materials of suppositories, witepsol, macrogol, tween 61, cacao butter, laurin fat, and glycerogelatin may be used.

According to one embodiment of the present disclosure, the pharmaceutical composition may be administered to a subject in a conventional manner through intravenous, intraarterial, intraperitoneal, intramuscular, intraarterial, intraperitoneal, intrasternal, transdermal, intranasal, inhalation, topical, rectal, oral, intraocular, or intradermal routes.

The dosage of the active ingredient according to the present disclosure may vary depending on the condition and body weight of the subject, the type and severity of the disease, the form of the drug, and the route and duration of administration and may be appropriately selected by those skilled in the art, and the daily dose may be 0.01 mg/kg to 200 mg/kg, preferably 0.1 mg/kg to 200 mg/kg, and more preferably 0.1 mg/kg to 100 mg/kg. The administration may be conducted once a day or divided into several doses, but the scope of the present disclosure is not limited thereby.

In addition, the present disclosure provides a combination of antimicrobial agents, including berberine chloride and an aminoglycoside drug.

The aminoglycoside drug may be selected from the group consisting of amikacin, kanamycin, and gentamicin, but is not limited thereto.

The combination of antimicrobial agents may alleviate or ameliorate an ototoxic hearing loss caused by an aminoglycoside drug.

In addition, the present disclosure provides a health functional food composition for preventing or ameliorating an ototoxic hearing loss caused by an aminoglycoside drug, including berberine chloride as an active ingredient.

The aminoglycoside drug may be selected from the group consisting of amikacin, kanamycin, and gentamicin, but is not limited thereto.

The health functional food may contain various nutritional supplements, vitamins, minerals (electrolytes), flavor agents such as synthetic flavor agents and natural flavor agents, coloring agents and thickening agents (cheese, chocolate, etc.), pectic acid and salts thereof, alginate and salts thereof, organic acids, protective colloidal thickeners, pH regulators, stabilizers, preservatives, glycerin, alcohol, and carbonating agents used in carbonated beverages.

In addition, it may contain pulp for the manufacture of natural fruit juices, synthetic fruit juices, and vegetable drinks. These ingredients may be used independently or in combination. In addition, the health functional food composition may be in any form of meat, sausage, bread, chocolate, candy, snacks, confectionery, pizza, instant noodles, chewing gum, ice cream, soups, beverages, teas, functional waters, drinks, alcohol, and vitamin complexes.

In addition, the health functional food may further include food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

Here, the content of active ingredients added to the food in the process of manufacturing the health functional food may be appropriately adjusted as needed, and preferably it may be added to be included in an amount of 1 part by weight to 90 parts by weight per 100 parts by weight of food.

### MODES FOR CARRYING OUT THE INVENTION

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### [Example 1] Organotypic Cochlear Explants

Three-day-old ICR (Institute for Cancer Research) mice were purchased from Hyochang Science (Daegu, South Korea) for cochlear implantation. Dissected cochleae were cultured in high-glucose Dulbecco's Modified Eagle's Medium (DMEM; Hyclone, Logan, UT, USA) containing 10% fetal bovine serum (Hyclone) and ampicillin (10 μg/mL; Life Technologies, Carlsbad, CA, USA) at 37° C. in a humidified atmosphere in the presence of 5% CO₂. After 16 hours of culture, cochleae were first treated with 0.1 μM or 1 μM BC for 1 hour, followed by addition of one of three aminoglycoside antibiotics (1.2 mM KM, 3 mM AK, or 100 μM GM) to the culture medium. BC concentrations were optimized for each aminoglycoside (0.1 μM for KM, 1 μM for AK and GM).

All animal experiments were approved by the Institutional Animal Care and Use Committee and followed the guidelines of the Animal Experiment Practice Committee of Kyungpook National University.

### [Example 2] Phalloidin Staining

To evaluate the morphology of inner hair cells (IHCs) and outer hair cells (OHCs) of the organ of Corti, the specimens were cultured for 48 hours and fixed with 4% paraformaldehyde (PFA, pH 7.4) in PBS for 15 minutes. After washing three times with PBS, a stereociliary bundle of explants was stained with Alexa Fluor 488-or 555-conjugated phalloidin (1:1,000; Invitrogen-Molecular Probes, Eugene, OR) in PBS for 1 hour at room temperature (RT). Specimens were mounted on glass slides using Fluoromount (Sigma-Aldrich, St. Louis, MO) and visualized using an Axio Imager A2 fluorescence microscope (Carl Zeiss, Oberkochen, Germany).

### [Example 3] Immunohistochemistry and Terminal Deoxynucleotidyl Transferase dUTP Nick End Labeling (TUNEL) Assay

Immunohistochemistry and TUNEL assay for active caspase-3 were used to determine whether cell death in the organ of Corti occurred by apoptosis. Specimens were fixed with 4% PFA, kept permeabilized with 0.1% Triton X-100 in PBS (PBS-Tx) for 30 minutes at room temperature (RT), blocked with 5% normal goat serum for 1 hour at room temperature (RT), and cultured overnight with anti-caspase-3 antibody (1:1,000; Cell Signaling Technology, Bevery, MA) diluted in blocking solution.

To detect DNA fragmentation leading to cell death, a TUNEL assay kit (Promega, Madison, WI) was used according to the manufacturer's protocol. Specimens were fixed with 4% paraformaldehyde in PBS for 15 minutes at room temperature (RT) and treated with 0.1% PBS-Tx in 0.1% sodium citrate for 30 minutes at 37° C. Fragmented DNA from cells was stained using TUNEL working solution at 37° C. for 30 minutes. F-actin was labeled with Alexa Fluor 555-conjugated phalloidin in PBS-Tx for 3 hours at room temperature (RT) in the dark and visualized using a Zeiss Axio Imager A2 fluorescence microscope.

### [Example 4] Measurement of Mitochondrial Damage Caused by Antibiotic-Induced Oxidative Stress

To analyze AK or KM-induced mitochondrial oxidative stress, cochlear explants were examined by MitoSOX Red assay and JC-1 assay. Levels of mitochondrial reactive oxygen species (ROS) were detected by staining with 5 μM MitoSOX Red (Invitrogen-Molecular Probes) for 5 minutes in a humidified atmosphere at 37° C. in the presence of 5% CO₂. The specimen was washed with PBS and visualized using a Zeiss Axio Imager A2 fluorescence microscope.

The cationic fluorescent dye MitoProbe™ JC-1 (Invitrogen-Molecular Probes) was used to measure the mitochondrial membrane potential. After 30 hours of culture, the specimens were washed with PBS and stained with JC-1 (2 μM) for 2 hours at 37° C. in the presence of 5% CO₂ in a darkroom. The sensitivity of the JC-1 assay was verified by adding 50 μM carbonyl cyanide 3-chlorophenylhydrazone (CCCP) to untreated specimens before JC-1 staining, and then the specimens were cultured at 37° C. in the presence of 5% CO₂ for 5 minutes for activation of CCCP. JC-1 fluorescence was split into red and green, and then the segmented images were converted to grayscale, followed by evaluation using ImageJ software (http://imageJ.nih.gov/ij/).

[Example 5] Quantification of hair cells and statistical analysis For quantitative analysis of hair cell damage, IHCs and OHCs with intact v-shaped stereopsis were individually counted along a 200 μm length of basilar membrane in the apical (30%), middle (50%), and basal (70%) regions of each cochlear probe. All experiments were performed independently at least three times. Data were analyzed using a two-tailed Student's t-test, with a P-value <0.05 considered statistically significant.

### [Experimental Example 1] Protective Effect of BC Against Drug-Induced Hair Cell Damage

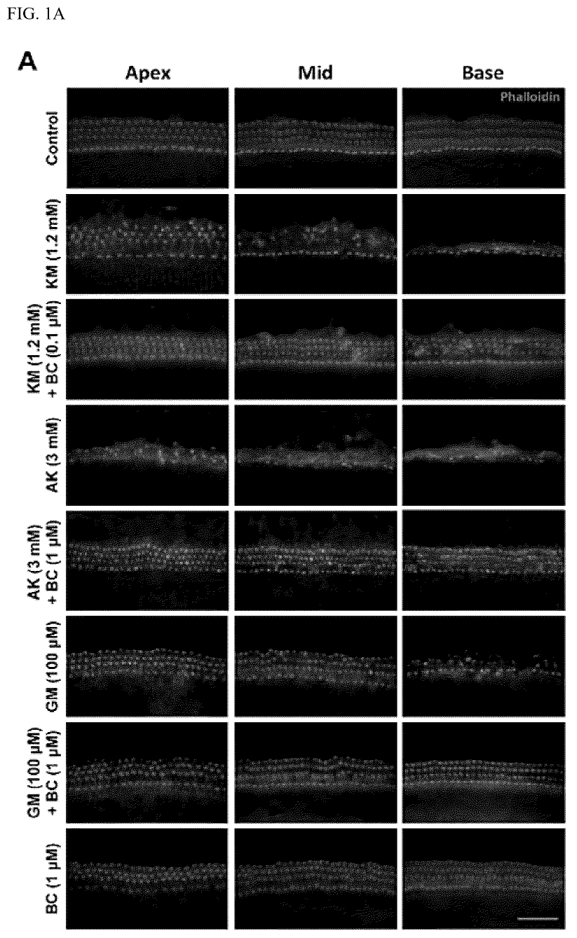

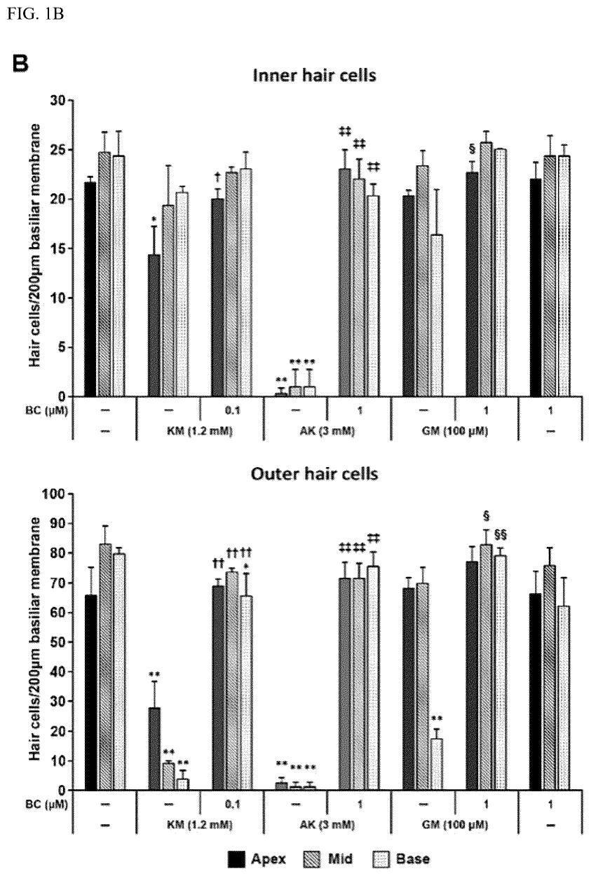

To determine whether BC protects hair cells, comparison was made on the severity of antibiotic-induced hair cell damage between BC-treated and untreated cochlear explants. In the untreated control group, IHCs and OHCs with well-shaped stereocilia showed intact arrangement from the apical to the basal cochlear turn. However, antibiotic treatment disrupted the arrangement of stereocilium and hair cells (FIG. 1A). KM not only altered the ciliary morphology of hair cells but also disrupted cell arrangement, with induction of cell loss in a complex manner. These changes were more severe in OHC than in IHC. AK-treated hair cells had most of their cilia disintegrated and lost, making them unrecognizable. Similar to KM, GM caused more severe damage to OHCs than IHCs, with loss of hair cells. All three antibiotics caused severe hair cell damage that increased in severity from apical to basal, which was consistent with the loss in the typical high-frequency range of ototoxic loss. However, treatment with 0.1 or 1 μM BC before antibiotic (KM, AK, or GM) treatment alleviated stereocilia degeneration and hair cell loss in all cochlear regions, providing a protective effect of BC against antibiotic-induced cytotoxicity in hair cells.

The protective effect of BC against hair cell damage caused by KM, AK, or GM was statistically determined in apical, middle and basal turns which are three cochlear regions, by counting surviving hair cells with V-shaped stereocilia (FIG. 1B). The cytotoxicity of BC was measured, and it was found that there are no detrimental effects on IHC or OHC. IHC was virtually destroyed by AK but was more resistant to KM and GM-induced cytotoxicity. Compared to IHC, OHC was significantly more susceptible to antibiotic-induced cytotoxicity. Importantly, it was found that, by pretreating both IHC and OHC with BC, antibiotic-induced damage was reduced to near-normal levels. Therefore, BC could effectively prevent antibiotic-induced hair cell loss in cochlear tissues.

### [Experimental Example 2] Inhibition of Apoptosis by BC in Mouse Cochlear Explants

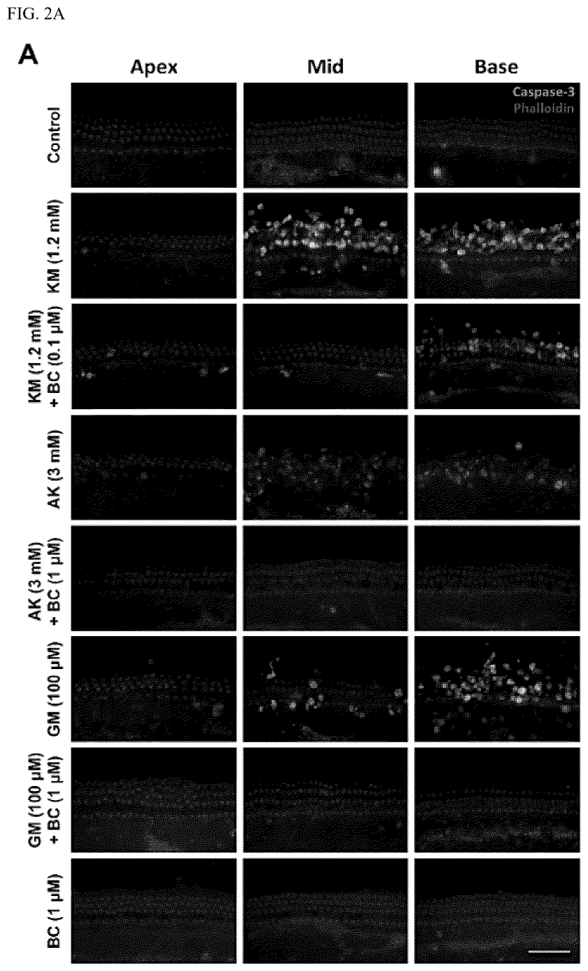

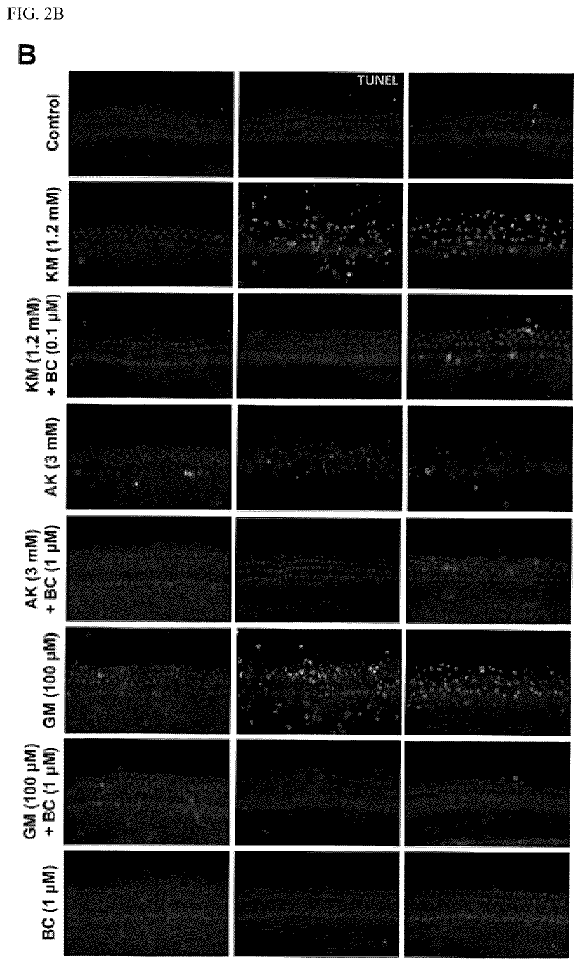

Because BC attenuated antibiotic-induced ototoxicity in mouse cochlear explants, examination was conducted on whether this effect was due to inhibition of apoptotic hair cell death. Two hallmarks of intrinsic apoptosis, caspase-3 activation and DNA fragmentation, were tested by immunohistochemical staining. Organs of Corti treated with KM, AK, or GM showed significantly increased levels of cleaved caspase-3 compared to untreated controls, whereas pre-treatment with BC before antibiotics reduced cleaved caspase-3 (FIG. 2A). Similarly, apoptotic DNA fragmentation in cells of the organ of Corti, as measured by TUNEL labeling, showed significant reduction by BC pretreatment to near control levels compared with KM, AK, or GM treatment (FIG. 2B). In both analyses, the signal intensities of cleaved caspase-3 and fragmented DNA were stronger toward the basal turn, consistent with the pattern of cell loss and ciliary damage as shown in FIG. 1. Therefore, these results indicated that hair cell damage induced by KM, AK, or GM in the mouse cochlea resulted in cell death, which was effectively suppressed by BC.

### [Experimental Example 3] Prevention of Oxidative Stress and Mitochondrial Damage by BC in Mouse Cochlear Hair Cells Induced by Aminoglycoside Drugs

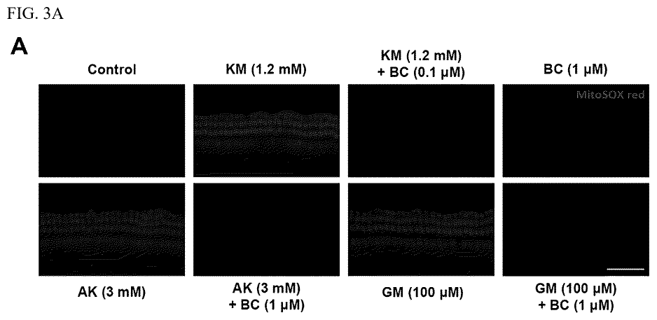

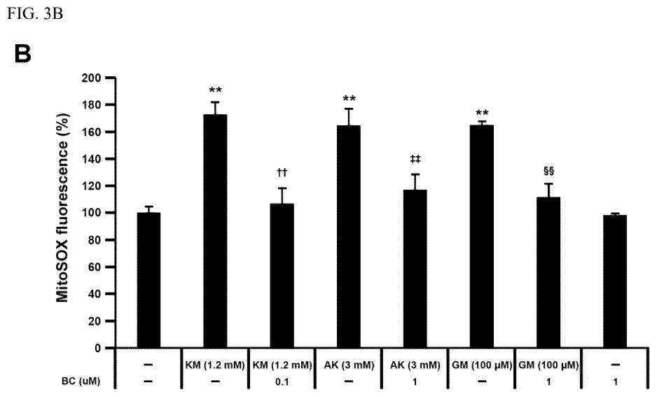

Because excessive accumulation of intracellular ROS leads to mitochondrial dysfunction, measurement was made on the accumulation of mitochondrial ROS induced by aminoglycoside drugs using MitoSOX Red fluorescence assay. According to FIG. 3A, when all three drugs were treated, excessive accumulation of ROS all occurred, resulting in higher fluorescence intensity. In contrast, a significant decrease in fluorescence intensity was observed in the BC pretreatment group. The fluorescence intensity with the integrated density of red fluorescence reduced in the BC treatment group was statistically significant compared to the AK, KM, and GM groups (FIG. 3B). This suggests that BC suppresses mitochondrial ROS accumulation in hair cells, thereby inhibiting cell death caused by oxidative stress.

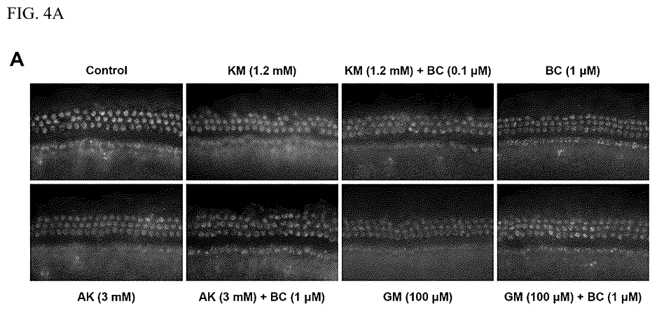

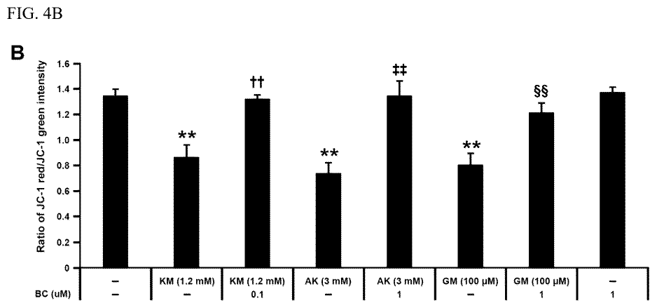

Mitochondria are a major source of intracellular ROS, but are also known to be vulnerable to ROS damage. Since depolarization of mitochondrial membrane potential causes mitochondrial dysfunction, comparison was made on mitochondrial membrane potential of cochlear explants treated with aminoglycoside drugs with or without BC treatment via MitoProbe™ JC-1 analysis. JC-1 monomers, which emit green fluorescent signals, have the characteristic of aggregating in mitochondria and producing red fluorescence in mitochondria with high membrane potential. Therefore, mitochondrial membrane potential was measured in a red/green fluorescence ratio. As a result, according to FIG. 4A, the hair cells treated with only KM, AK, or GM showed a significant decrease in the red/green ratio along with a loss of red signal, indicating mitochondrial damage induced by aminoglycosides. However, the red/green fluorescence ratio was found to increase in BC-pretreated hair cells, suggesting that the protective effect of BC was statistically significant (n=3 per group, *P<0.05 and **P<0.01; FIG. 4B). These results indicated that BC prevented aminoglycoside-induced loss of mitochondrial membrane potential.

The foregoing description herein is for illustrative purposes only, and it will be appreciated by those skilled in the art to which the present disclosure pertains that the present disclosure may be easily modified into other specific forms without altering the technical idea or essential features of the present disclosure. Therefore, it should be understood that the examples set forth above are exemplary in all respects and not limiting.

The scope of the present disclosure is indicated by the claims described below, and all changes or modifications derived from the meaning and scope of the claims and their equivalent concepts should be interpreted as being included in the scope of the present disclosure.

1. A method of preventing or treating an ototoxic hearing loss caused by an aminoglycoside drug, comprising:
administering a pharmaceutical composition comprising berberine chloride as an active ingredient to a subject.

2. The method of claim 1, wherein the aminoglycoside drug is selected from the group consisting of amikacin, kanamycin, and gentamicin.

3. The method of claim 1, wherein the pharmaceutical composition inhibits apoptosis of auditory cells and increases the number of hair cells.

4. The method of claim 1, wherein the pharmaceutical composition inhibits accumulation of mitochondrial reactive oxygen species (ROS) in hair cells.

5. A combination of antimicrobial agents, comprising berberine chloride and an aminoglycoside drug.

6. The combination of antimicrobial agents of claim 5, wherein the aminoglycoside drug is selected from the group consisting of amikacin, kanamycin, and gentamicin.

7. The combination of antimicrobial agents of claim 5, wherein the combination of antimicrobial agents alleviates or ameliorates an ototoxic hearing loss caused by an aminoglycoside drug.

8. A method of preventing or ameliorating an ototoxic hearing loss caused by an aminoglycoside drug, comprising:
administering a health functional food composition comprising berberine chloride as an active ingredient to a subject.

9. The method of claim 8, wherein the aminoglycoside drug is selected from the group consisting of amikacin, kanamycin, and gentamicin.
